(19) United States
(12) Patent Application Publication
(10) Pub. No.: US 2009/0281111 A1
(43) Pub. Date: Nov. 12, 2009

(54) 3-HYDROXY GEPIRONE FOR THE TREATMENT OF ATTENTION DEFICIT DISORDER AND SEXUAL DYSFUNCTION

(75) Inventors: Stephen J. KRAMER, Houston, TX (US); Louis F. Fabre, Houston, TX (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: Fabre-Kramer Pharmaceuticals, Inc., Houston, TX (US)

(21) Appl. No.: 12/437,180

(22) Filed: May 7, 2009

Related U.S. Application Data
(60) Provisional application No. 61/051,555, filed on May 8, 2008.

(51) Int. Cl. A61K 31/497

(52) U.S. Cl. 514/252.18

(57) ABSTRACT

The present invention relates to a method for alleviation, prevention, and treatment of attention deficit disorder, sexual dysfunction, and related conditions by administering certain bioactive metabolites of the known anti-depressant compound gepirone. In a preferred embodiment, the compound is 4,4,-dimethyl-3-hydroxy-1-[4-[4-(2-pyrimidinyl)-1-piperazinyl]butyl]-2,6-piperidinedione (3-OH gepirone).

### BACKGROUND OF THE INVENTION

1. Field of the Invention

The present invention relates to a method for alleviation, prevention, and treatment of attention deficit disorder, sexual dysfunction, and related conditions by administering certain bioactive metabolites of the known anti-depressant compound gepirone. In a preferred embodiment, the compound is 4,4,-dimethyl-3-hydroxy-1-[4-[4-(2-pyrimidinyl)-1-piperazinyl]butyl]-2,6-piperidinedione (3-OH gepirone), however other gepirone metabolites and combinations thereof are possible and contemplated. Surprisingly, these bioactive metabolites of gepirone show improved bioavailability characteristics and improved potential for immediate action and long-term treatment regimens when compared to gepirone and other therapeutic azapirones. Accordingly, the invention provides new and improved methods for treating a variety of psychological disorders and conditions.

2. Description of the Related Art

Attention Deficit Disorder

Attention deficit disorder (ADD) is a learning disorder that relates to developmentally inappropriate inattention and impulsivity. ADD may also be referred to as disruptive behavior disorder or minimal brain dysfunction. ADD may be present with or without hyperactivity. A common disorder, ADD accounts for more child mental health referrals than any other single disorder. ADD is estimated to affect 3 to 5% of school-aged children, and is much more frequent in males than in females, with a male-to-female ratios ranging from 4:1 to 9:1. On the average, at least one child in every classroom in the United States needs help for the disorder. ADD often continues into adolescence and adulthood, and can cause a lifetime of frustrated dreams and emotional pain. In addition, ADD may affect the behavior of children at any cognitive level.

ADD is a diagnosis applied to children and adults who consistently display certain characteristic behaviors over a period of time. The most common behaviors fall into two categories: inattention and impulsivity. Attention deficit disorder with hyperactivity is diagnosed when the signs of overactivity are obvious. Inappropriate inattention causes increased rates of activity and impersistence or reluctance to participate or respond. A subject suffering from ADD exhibits a consistent pattern of inattention and/or hyperactivity-impulsivity that is more frequent and severe than is typically observed in individuals at a comparable level of development. Such subjects must suffer clear evidence of interference with developmentally appropriate social, academic, or occupational functioning.

Although subjects with ADD and without hyperactivity may not manifest high activity levels, most exhibit restlessness or jitteriness, short attention span, and poor impulse control. These are qualitatively different from those seen in conduct and anxiety disorders. Inattention is described as a failure to finish tasks started, easy distractibility, seeming lack of attention, and difficulty concentrating on tasks requiring sustained attention. Impulsivity is described as acting before thinking, difficulty taking turns, problems organizing work, and constant shifting from one activity to another. Impulsive responses are especially likely when involved with uncertainty and the need to attend carefully. Hyperactivity is featured as difficulty staying seated and sitting still, and running or climbing excessively. A more complete description of the symptoms and diagnostic criteria of attention deficit disorder with or without hyperactivity are provided by DSM-IV (Diagnostic and Statistical Manual of Mental Disorders, 1994; 78-85), which is incorporated herein by reference.

No single treatment has been completely effective for attention deficit disorder. Psychostimulant medications combined with behavioral and cognitive therapies (e.g., selfrecording, self-monitoring, modeling, and role-playing) have the greatest controlling influence on symptom expression. Used alone, medication has been effective predominantly with less aggressive ADD children coming from stable home environments. Elimination diets, megavitamin treatments, psychotherapy, and biochemical interventions (e.g., the administration of neurochemicals) have had only minor, unsustained effects.

For decades, medications have been used to treat the symptoms of ADD. The three most common medications in both adults and children are the stimulants; methylphenidate (RITALIN™), dextroamphetamine (DEXEDRINE™ or DEXTROSTAT™), and pemoline (CYLERT™). For many people, these medicines dramatically reduce their hyperactivity and improve their ability to focus, work, and learn. The medications may also improve physical coordination, such as handwriting and ability in sports. Recent research by National Institute of Mental Health suggests that these medicines may also help children with an accompanying conduct disorder to control their impulsive, destructive behaviors. Nine out of 10 children improve on one of these three stimulant drugs.

Different doctors use the medications in slightly different ways. CYLERT™ is available in one form, which naturally lasts 5 to 10 hours. RITALIN™ and DEXEDRINE™ come in short-term tablets that last about 3 hours, as well as longer-term preparations that last through the school day.

Stimulant drugs, when used with medical supervision, are usually considered safe. However, a common problem with stimulant drugs is that they can be addictive to teenagers and adults if misused. While on these medications, some children may lose weight, have less appetite, and temporarily grow more slowly. Others may have problems falling asleep. Some doctors believe that stimulants may also make the symptoms of Tourette's syndrome worse.

The most commonly prescribed ADD medication is RITALIN™, which is generally more effective than tricyclic antidepressants (e.g., IMIPRAMINE™), caffeine, and other psychostimulants (e.g., PEMOLINE™ and DEANOL™) and has fewer side effects than dextroamphetamine. Common side effects of RITALIN™ are sleep disturbances (e.g., insomnia), depression or sadness, headache, stomachache, suppression of appetite, elevated blood pressure, and, with large continuous doses, a reduction of growth. Long-term benefits of medication with RITALIN™, however, have not been demonstrated conclusively. Some research indicates that use of medication permits participation in activities previously inaccessible because of poor attention and impulsivity. The frequency of side effects, potential addictiveness, and limited success of stimulant drugs has led to a search for alternate means of treating or preventing attention deficit disorders.

### Sexual Dysfunction

Sexual Dysfunction may be defined as difficulty during any stage of the sexual act (which includes desire, arousal, orgasm, and resolution) that prevents the individual or couple from enjoying sexual activity. Sexual dysfunction disorders are generally classified into four categories: sexual desire disorders, sexual arousal disorders, orgasm disorders, and sexual pain disorders. Sexual dysfunction is discussed in U.S. Patent Publication No. 2007/0123536, herein incorporated by reference in its entirety.

Sexual desire disorders (decreased libido) may be caused by a decrease in the normal production of estrogen (in women) or testosterone (in both men and women). Other causes may be aging, fatigue, pregnancy, and medications (such as anti-depressants such as fluoxetine, sertraline, and paroxetine are well known for reducing desire in both men and women. Psychiatric conditions, such as depression and anxiety, can also cause decreased libido.

Sexual arousal disorders were previously known as frigidity in women and impotence in men. These have now been replaced with less judgmental terms. Impotence is now known as erectile dysfunction, and frigidity is now described as any of several specific problems with desire, arousal, or anxiety. For both men and women, these conditions may appear as an aversion to, and avoidance of, sexual contact with a partner. In men, there may be partial or complete failure to attain or maintain an erection, or a lack of sexual excitement and pleasure in sexual activity.

Orgasm disorders are a persistent delay or absence of orgasm following a normal sexual excitement phase. The disorder occurs in both women and men. Again, the SSRI antidepressants are frequent culprits—these may delay the achievement of orgasm or eliminate it entirely.

Sexual pain disorders affect women almost exclusively, and are known as dyspareunia (painful intercourse) and vaginismus (an involuntary spasm of the muscles of the vaginal wall, which interferes with intercourse). Dyspareunia may be caused by insufficient lubrication (vaginal dryness) in women.

Sexual dysfunctions are more common in the early adult years, with the majority of people seeking care for such conditions during their late 20s through 30s. The incidence increases again in the geriatric population, typically with gradual onset of symptoms that are associated most commonly with medical causes of sexual dysfunction. Sexual dysfunction is more common in people who abuse alcohol and drugs. It is also more likely in people suffering from diabetes and degenerative neurological disorders. Ongoing psychological problems, difficulty maintaining relationships, or chronic disharmony with the current sexual partner may also interfere with sexual function.

Symptoms of sexual dysfunction may include loss of libido, inability to feel aroused, painful intercourse in both male and female patients. In men, symptoms may include inability to attain or maintain an erection, delay or absence of ejaculation, and inability to control timing of ejaculation. In women, symptoms may include inability to relax vaginal muscles enough to allow intercourse, inadequate vaginal lubrication before and during intercourse, inability to attain orgasm, and burning pain on the vulva or in the vagina with contact to those areas.

Sexual dysfunction is common among individuals with depression. Depressed individuals show decreased sexual interest and reported reduced levels of arousal. Sexual dysfunction is also a common side effect of antidepressant treatment, particularly pharmacotherapy with serotonin reuptake inhibitors (SRIs). The sexual response cycle consists of 4 phases: desire, arousal, orgasm, and resolution. All of these phases may be affected by reproductive hormones and neurotransmitters. Estrogen, testosterone, and progesterone promote sexual desire. Dopamine promotes desire and arousal. Norepinephrine promotes arousal. Prolactin inhibits arousal. Oxytocin promotes orgasm. Serotonin may have a negative impact on the desire and arousal phases of the sexual response cycle, possibly due to its inhibition of dopamine and norepinephrine.

Treatment of sexual dysfunction involves identifying the specific cause and, often, treating the underlying condition. Medical causes that are reversible or treatable are usually managed medically or surgically. Physical therapy and mechanical aides may prove helpful for some people experiencing sexual dysfunction due to physical illnesses, conditions, or disabilities.

Neurological and psychological factors play an important role in sexual dysfunction. Anxiety, fear, and depression can particularly be addressed with treatments such as psychological therapy and medications. Dopamine is known to promote desire and arousal. Accordingly, dopaminergic agents may be helpful for the treatment of antidepressant-induced sexual dysfunction.

Therapy for the treatment of ADD and/or sexual dysfunction may be to target the dopaminergic system. Deregulation of the dopaminergic system has been linked with each of these conditions. Such deregulation has also been linked with Parkinson's disease, Tourette's syndrome, schizophrenia, attention deficit hyperactive disorder (ADHD) and generation of pituitary tumours (Vallone et al, Neurosci Biobehav Rev 2000 January; 24:125-32). The azapirone, buspirone, has been speculated as having a potential therapeutic role in treatment of ADHD (Balon, J. Clin. Psychopharma. 1990; 10: 77, and Malhotra et al, J. Am. Acad. Child Adolesc. Psychiatry 1998; 57: 364-371).

Buspirone exhibits an affinity for a series of receptors including serotonin receptors, dopamine receptors, and α-adrenergic receptors. The effect of buspirone on the dopaminergic system occurs by enhancing dopamine synthesis and release (Tunnicliff et al, Neuropharmacology 1992; 31: 991-5). Buspirone blocks the presynaptic dopaminergic receptors rather than the postsynaptic dopaminergic receptors, thereby increasing the firing of the midbrain neurons and blocking the inhibiting effects of γ-aminobutyric acid on dopaminergic neurons in the zona compacta of the substantia nigra (Eison and Temple, Am. J. Med. 1986; 80(3B suppl): 1-9). Although the serotonergic activity may be related to improved behavior and impulsivity, according to Balon and Malhotra et al, the dopaminergic activity of buspirone leads to improved attention span and decreased hyperactivity associated with ADHD. However, more recent reports on treatment strategies of attention deficit disorder have suggested that buspirone may have a potentially deleterious effect on patients having ADHD (Popper, Child Adolesc Psychiatr Clin NAm 2000; 9: 605-46). Busprone may be expected to have similar deleterious effects on patients with other conditions linked to the dopaminergic system.

Accordingly, there remains a critical need for novel treatment strategies of patients suffering from ADD, with or without hyperactivity, and sexual dysfunction. Moreover, there remains a critical need for treatment strategies, which are safe and effective with a reduction in or elimination of any side effects associated with existing treatment strategies.

### SUMMARY OF THE INVENTION

Accordingly, it is an object of the present invention to provide methods for treatment of attention deficit disorder, with or without hyperactivity, anxiety, depression, and sexual dysfunction, or symptoms thereof. This object can be achieved by administering to a patient in need thereof with a 5-HT_{1A} receptor agonist.

The present invention is based, in part, on the discovery that a patient suffering from attention deficit disorder can be treated by an azapirone 5-HT_{1A} receptor agonist, which lacks dopamine receptor activity. Accordingly, it is an object of the present invention to treat a patient having attention deficit disorder, with or without hyperactivity, or symptoms thereof, with an azapirone 5-HT_{1A} receptor agonist, which lacks dopamine receptor activity. Examples of suggested azapirone 5-HT_{1A} receptor agonists, which lack dopamine receptor activity, include gepirone, ipsapirone, and tandospirone.

One of the more important azapirones is gepirone, which has the following structure:

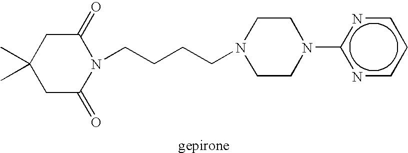

Gepirone has been used to effectively treat anxiety disorders and depression (Casacalenda, Canadian J. of Psychiatry, 43:722-730 (1998)). However, it has several drawbacks from the standpoint of an ideal therapeutic anxiolytic or anti-depressant. It has low bioavailability characteristics when delivered orally, on the order of 14-18%. In addition, the half-life of gepirone is very short. As a result, an extended release formulation of gepirone is preferred so that sustained therapeutic levels can be delivered during a standard regimen without increasing dosage levels. Furthermore, in a small percentage of cases, gepirone has been associated with side effects such as nausea and vomiting. Accordingly, 5-HT.sub.1A agonists with improved properties and characteristics are still in need.

Certain bioactive metabolites of gepirone, especially 4,4,-dimethyl-3-hydroxy-1-[4-[4-(2-pyrimidinyl)-1-piperazinyl]butyl]-2,6-piperidinedione (3-OH gepirone) have been found to be agents useful in treating anxiety, depression, and a number of other psychological disorders. 3-OH gepirone has the following structural formula:

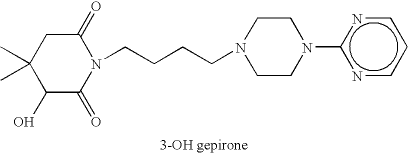

Examples of other bioactive gepirone metabolites are listed below:

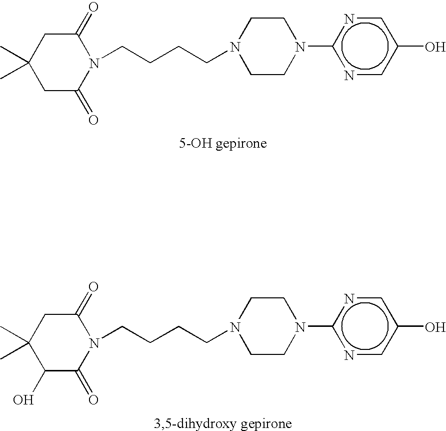

The bioactive gepirone metabolites of this invention include those compounds listed above that can be used to treat psychological disorders, or that functionally interact with a 5-HT_{1A} receptor. A bioactive gepirone metabolite includes any active salt form, hydrate form, enantiomeric form or mixture, or crystal form of the compound. Preferably, the bioactive gepirone metabolite is 3-OH gepirone. 3-OH Gepirone is discussed in U.S. Pat. No. 6,534,507, herein incorporated by reference in its entirety.

In one embodiment, the 5-HT_{1A} receptor agonist is administered in conjunction with an agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

In another embodiment, the 5-HT_{1A} receptor agonist is administered with the administration of methylphenidate (RITALIN™).

In another embodiment, the 5-HT_{1A} receptor agonist is administered with a pharmaceutically acceptable carrier.

In another embodiment, the 5-HT_{1A} receptor agonist is administered orally, rectally, nasally, parenterally, intracistemally, intravaginally, intraperitoneally, sublingually, topically, or bucally.

In another embodiment, the therapeutically effective amount of the 5-HT_{1A} receptor agonist is similar to the anxiolytic dose of the medication, e.g.: 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day) in single or multiple doses.

In another embodiment, the patient in need thereof also suffers from one or more of anxiety, depression, obesity, drug abuse/addiction, alcohol abuse, sleep disorders, TIC disorder, and behavioral/cognitive symptoms of Alzheimer's disease.

Another object of the present invention is to provide a treatment regimen of concurrently administering to a patient in need thereof mixtures of two or more of the compounds of the present invention.

Yet another object of the present invention is to provide a treatment regimen of administering to a patient in need thereof a single or divided dose of a first compound followed by, on the same day or a subsequent day, a single or divided dose of one or more additional compounds.

The above object highlights certain aspects of the invention. Additional objects, aspects and embodiments of the invention are found in the following detailed description of the invention.

### BRIEF DESCRIPTION OF THE DRAWINGS

A more complete appreciation of the invention and many of the attendant advantages thereof will be readily obtained as the same becomes better understood by reference to the following Figures in conjunction with the detailed description below.

FIG. 1 depicts a chromatograph of bioactive gepirone metabolites isolated from a plasma sample: a large peak of 3-OH gepirone (labeled), 5-OH gepirone (peak 2), and 5-Me-OH gepirone (peak 1).

FIG. 2 is a table showing the time course of plasma levels of 3-OH-gepirone present in plasma (ng/ml) after administration of gepirone to human subjects. “Time (H)” represents time after administration.

### DETAILED DESCRIPTION OF THE PREFERRED EMBODIMENTS

Unless specifically defined, all technical and scientific terms used herein have the same meaning as commonly understood by a skilled artisan in organic chemistry, biochemistry, psychology, psychiatry, medicine, neurochemistry, and neurology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

As used herein, the general term “attention deficit disorder” includes attention deficit disorder and disruptive behavior disorder each of which may be present with or without hyperactivity.

As improved 5-HT_{1A} agonists, 3-OH gepirone and the other bioactive gepirone metabolites can be used in methods to alleviate a number of psychological disorders. Preferred methods alleviate depression, anxiety, generalized anxiety disorder, panic disorder, obsessive compulsive disorder, alcohol abuse, addiction, atypical depression, infantile autism, major depressive disorder, depression with melancholia, premenstrual syndrome, and attention deficit hyperactivity disorder or symptoms of these disorders. The method comprises administering an effective amount of the bioactive gepirone metabolite, or a pharmaceutically acceptable salt or hydrate thereof, to a mammal. Preferably, the bioactive gepirone metabolite of these methods is selected from the group consisting of 3-OH gepirone, 3,5-dihydroxy gepirone, and 5-OH gepirone. The method may employ any one of these compounds. However, combinations of these metabolites, or combinations of the metabolites with other active or inert ingredients, are also contemplated.

The invention concerns methods for ameliorating depression, anxiety, or psychological disorders in a mammal in need of such treatment, which comprises administering to the mammal an effective amount or dose of a bioactive gepirone metabolite such as 3-OH gepirone. As used herein, the administration of a bioactive gepirone metabolite includes the administration of any active salt form, hydrate form, enantiomeric form or mixture, or crystal form of the compound. An effective oral dose should, in general, be in the range of from about 0.1 to 2 mg per kg of body weight. Alternatively, the effective dose or delivery system should result in plasma concentrations in the range of about 1 ng/ml to about 20 ng/ml, preferably about 1 ng/ml to about 5 ng/ml. The compounds like 3-OH gepirone can be administered via oral, sublingual, buccal, transdermal, rectal, or transnasal routes, thereby minimizing destructive first-pass metabolism. Systemic administration of 3-OH gepirone may be by a parenteral route, e.g. intramuscular, intravenous, subcutaneous, etc. Systemic administration may also be achieved by oral administration of a prodrug, a precursor or derivative form of 3-OH gepirone or gepirone metabolite. In this case the precursor or derivative form minimizes destructive metabolism of 3-OH gepirone or functions physiologically to release it into the mammal's system. One skilled in the art is familiar with methods to achieve this. In accordance with good clinical practice, it is preferable to administer 3-OH gepirone, or a precursor form, at concentration levels that will produce effective antidepressant and/or anxiolytic effects without causing harmful or untoward side-effects.

The invention also concerns compositions comprising 3-OH gepirone, or 5-OH gepirone, or 3,5-dihydroxy gepirone, or a combination of any of them. Preferably, these compositions are prepared to be administered to a mammal. Administration to a mammal can be by any number of drug deliver routes (See, for example, Remington's Pharmaceutical Sciences, 18th Edition, Genero et al. eds., Easton: Mack Publishing Co. for a description of a variety of drug delivery technologies available to one skilled in the art for use here). Preferably, the delivery route is an oral formulation, a parenteral formulation, or a transdermal formulation.

Formulations comprising 3-OH gepirone, 5-OH gepirone, or 3,5-dihydroxy gepirone, or the bioactive metabolite of gepirone, can be given in a oral dosage forms or parenteral forms comprised of an effective antidepressant and/or anxiolytic amount of 3-OH gepirone, 5-OH gepirone, or 3,5-dihydroxy gepirone, or one of the pharmaceutically acceptable acid addition salts thereof, or a hydrate thereof, in a pharmaceutically acceptable carrier. A variety of carriers are known in the art. Pharmaceutical compositions that provide from about 5 to 50 mg of the active ingredient per unit dose are preferred and are conventionally prepared as tablets, pills, capsules, aqueous solutions, and aqueous or oily suspensions. 3-OH gepirone, 5-OH gepirone, and 3,5-dihydroxy gepirone can also be given orally when compounded in a precursor or prodrug form in an oral dosing formulation such as a tablet, lozenge, capsule, syrup, elixir, aqueous solution or suspension.

As discussed in U.S. Pat. No. 6,534,507, 3-OH gepirone has been found to be highly selective for the 5-HT_{1A} receptor among the serotonergic receptor subtypes. 3-OH gepirone appears to have only weak binding affinity for dopaminergic and alpha-adrenergic receptors. In this regard, 3-OH gepirone is more selective than gepirone. Further, gepirone and buspirone interact with the 5-HT₇ and 5-HT_{2A} receptors much more specifically and at lower concentrations than does 3-OH gepirone, meaning 3-OH gepirone demonstrates a better selectivity profile than the gepirone or buspirone. As a result, 3-OH gepirone possesses an improved side effect profile compared to gepirone and buspirone since the potential for interacting with receptors other than 5-HT_{1A} is markedly lower. Moreover, 3-OH gepirone shows insignificant dopaminergic binding. Similarly, 3-OH gepirone and the other compounds did not display significant affinity at alpha-adrenergic receptors with the exception of weak binding at the Alpha 2C receptor (Alpha 2A, Alpha 2B, and Alpha 2C tested). With regard to muscarinic receptor binding data, gepirone, 3-OH gepirone, and 1-pyrimidinylpiperazine do not exhibit any affinity for muscarinic receptors (M₁, M₂, M₃ or M₄), with pKi values below 4.34 for all four receptor subtypes. Treatment with 3-OH gepirone likely results in a superior side effect profile than the comparative buspirone and gepirone. In sum, the bioactive gepirone metabolites exemplified by 3-OH gepirone exhibit a selective binding profile indicative of compounds that can be used clinically for treatment of anxiety, depression, and other psychological disorders.

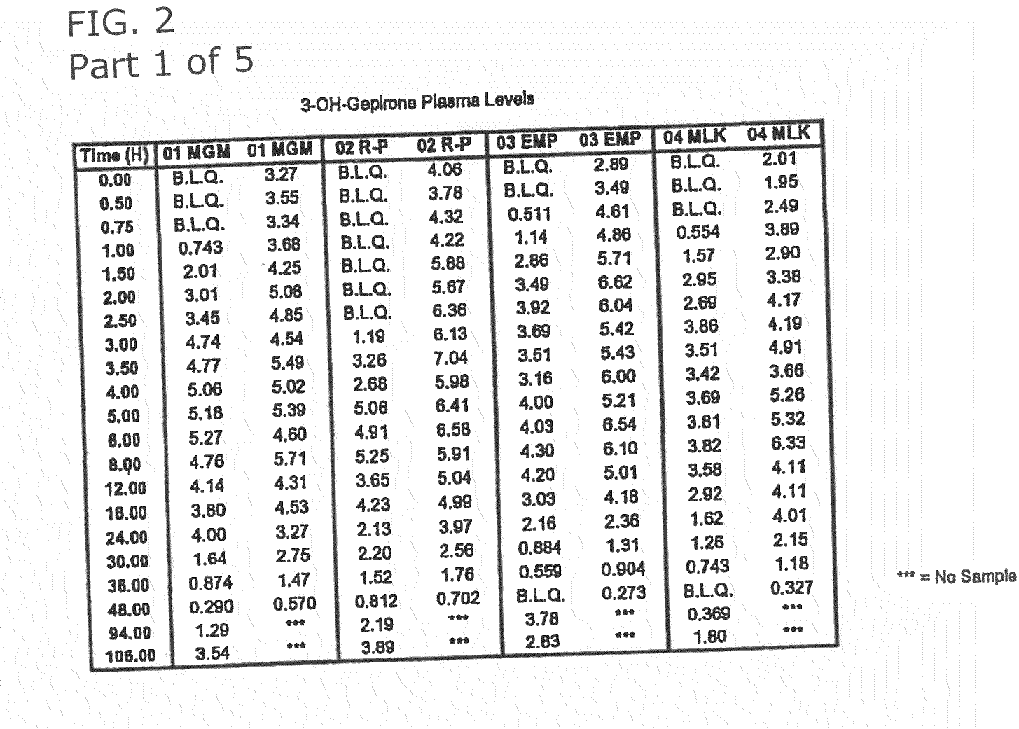

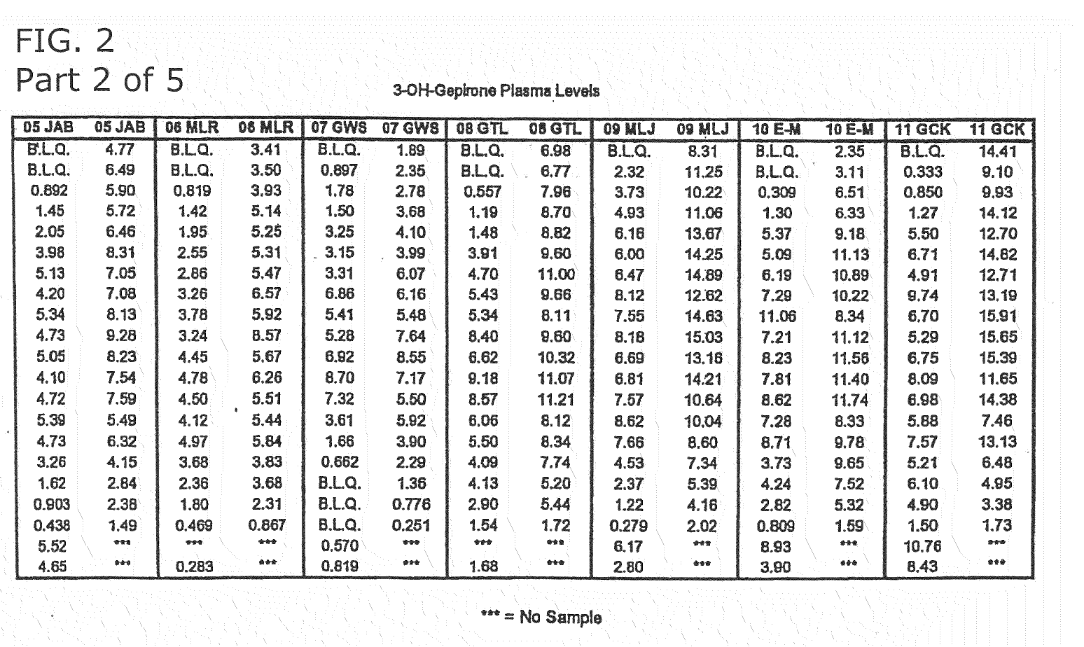

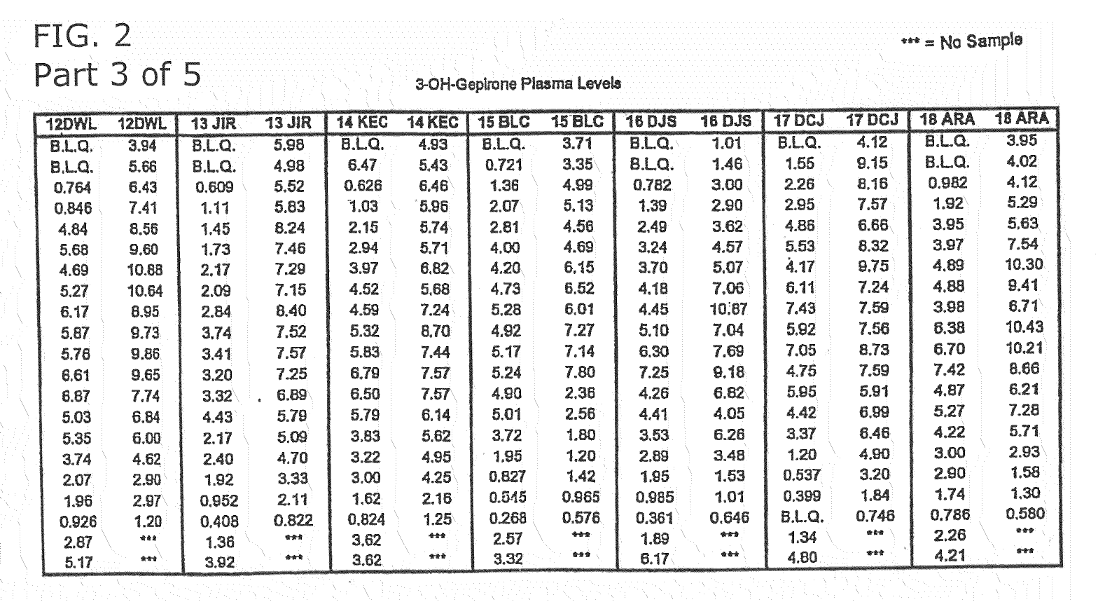

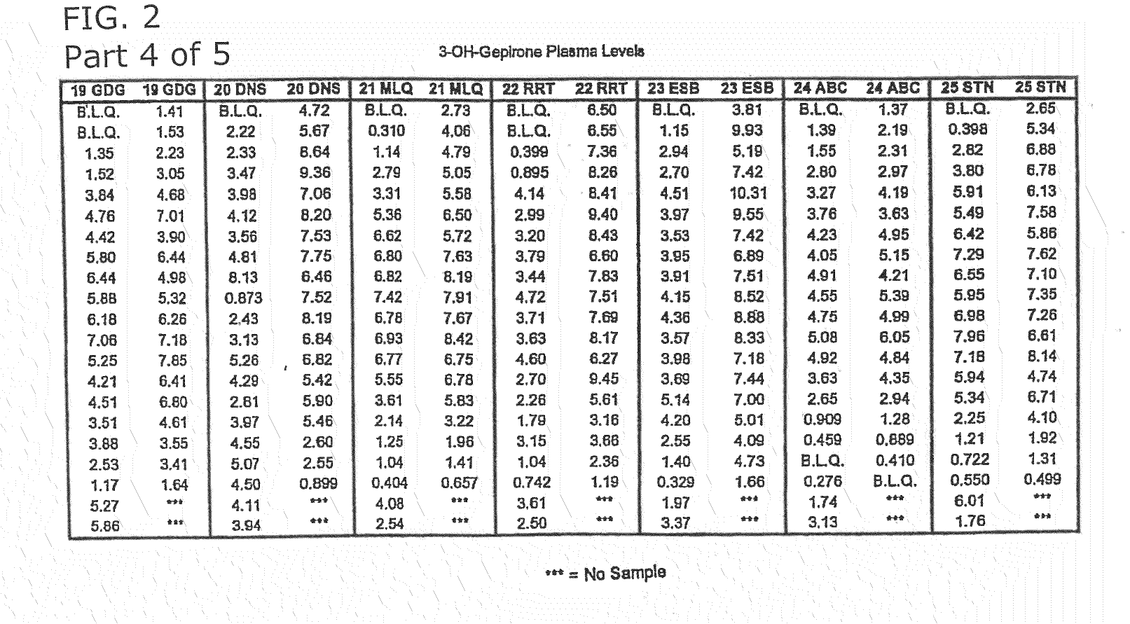

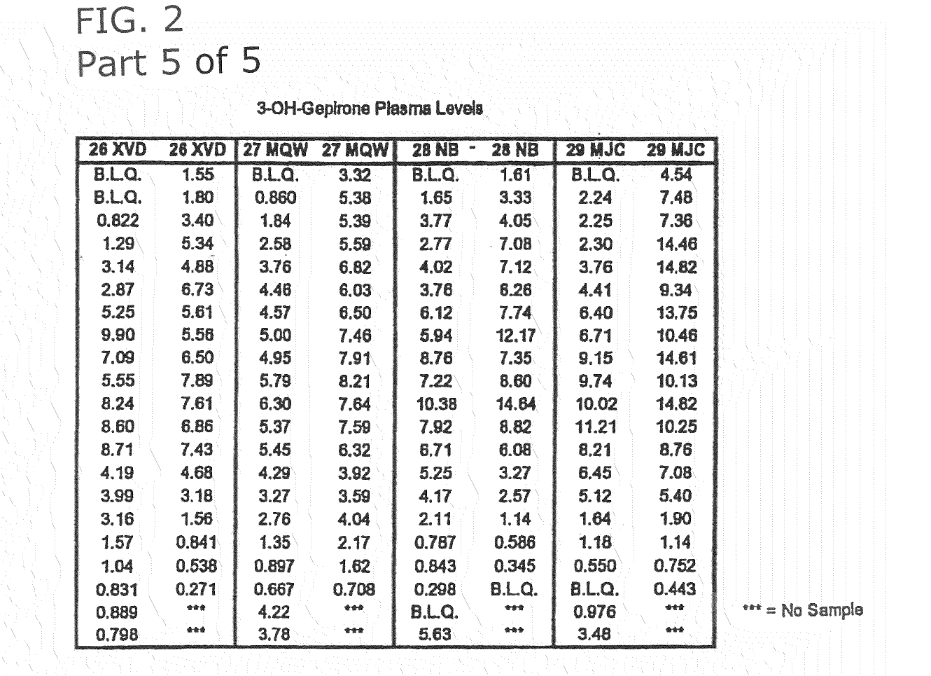

In addition, data shows that 3-OH gepirone will act as a much superior immediate action therapeutic compared to gepirone and buspirone. FIG. 2 depicts the plasma levels of 3-OH gepirone in a number of human subjects who were administered a dose of gepirone. Clearly, 3-OH gepirone is available quickly and persists in the plasma for extended periods of time. In contrast, both gepirone and buspirone have very short half-lives and low bioavailibilty profiles (about 1% for buspirone and 14-18% for gepirone). Without being limited by this theory, the inventors consider the additional-OH group on 3-OH gepirone compound and the other bioactive gepirone metabolites as affording an improved water solubility characteristic compared to gepirone and buspirone. This improved characteristic reduces the first-pass degradation of 3-OH gepirone in liver (see also Example 2 below).

Accordingly, the 3-OH gepirone compound and the similar bioactive gepirone metabolites possess superior properties compared to gepirone and buspirone when the compound is used in a pharmaceutical composition or for treating psychological disorders.

The pharmaceutically acceptable acid addition salts of 3-OH gepirone and the bioactive gepirone metabolites are also considered useful as antidepressant or anxiolytic agents or in treating psychological disorders. By definition, these are salts in which the anion does not contribute significantly to toxicity or pharmacological activity of the base form of 3-OH gepirone or the bioactive gepirone metabolite.

Acid addition salts are obtained by methods known in the art and can encompass a reaction of 3-OH gepirone or the bioactive gepirone metabolite with an organic or inorganic acid, preferably by contact in solution. Examples of useful organic acids are carboxylic acids such as maleic acid, acetic acid, tartaric acid, propionic acid, fumaric acid, isethionic acid, succinic acid, pamoic acid, and the like; useful inorganic acids are hydrohalide acids such as HCl, HBr, HI; sulfuric acid; phosphoric acid; and the like. An HCl acid salt of 3-OH gepirone is preferred.

As non-limiting examples, acid salts of the bioactive gepirone metabolites may also include: acetate, adipate, alginate, aspartate, benzoate, benzenesulfonate, bisulfate, butyrate, citrate, camphorate, camphorsulfonate, cyclopentanepropionate, digluconate, dodecylsulfate, methanesulfonate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts may also be employed and non-limiting examples of base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products can also be obtained.

Preferred oral compositions are in the form of tablets or capsules and in addition to 3-OH gepirone or a precursor form of 3-OH gepirone may contain conventional excipients such as binding agents (e.g., syrup, acacia, gelatin, sorbitol, tragecanth, or polyvinyl pyrrolidone), fillers (e.g., lactose, sugar, maize-starch, calcium phosphate, sorbitol or glycine), lubricants (e.g., magnesium stearate, talc, polyethyleneglycol or silica), disintegrants (e.g., starch), and wetting agents (e.g., sodium lauryl sulfate). Solutions or suspensions of 3-OH gepirone with conventional pharmaceutical vehicles are employed for parenteral compositions such as an aqueous solution for intravenous injection or an oily suspension for intramuscular injection. Such compositions having the desired clarity, stability and adaptability for parenteral use are obtained by dissolving from 0.1% to 10% by weight of the active ingredient (3-OH gepirone or a pharmaceutically acceptable acid addition salt or hydrate thereof) in water or a vehicle consisting of a polyhydric aliphatic alcohol such as glycerine, propylene glycol, and polyethylene glycols or mixtures thereof. The polyethylene glycols consist of a mixture of non-volatile, normally liquid, polyethylene glycols, which are soluble in both water and organic liquids and which have molecular weights from about 200 to 1500.

3-OH gepirone and the bioactive gepirone metabolites may also be prepared in a transdermal delivery method or other extended release delivery method (see U.S. Pat. Nos. 5,837,280, 5,633,009, and 5,817,331, each specifically incorporated herein by reference). One skilled in the art is familiar with numerous methods for designing and optimizing formulations and delivery methods to deliver the 3-OH gepirone and bioactive gepirone metabolites in effective and non-toxic ways. Remington's Pharmaceuticals Sciences, 18th Edition (specifically incorporated herein by reference), can be relied on and used for these purposes, especially Part 8 therein, “Pharmaceutical Preparations and Their Manufacture.”

3-OH gepirone may be synthesized by methods readily available in the chemical literature and known to one skilled in synthetic organic chemistry. One method of preparation utilizes gepirone as a starting material and the process is shown in Scheme 2.

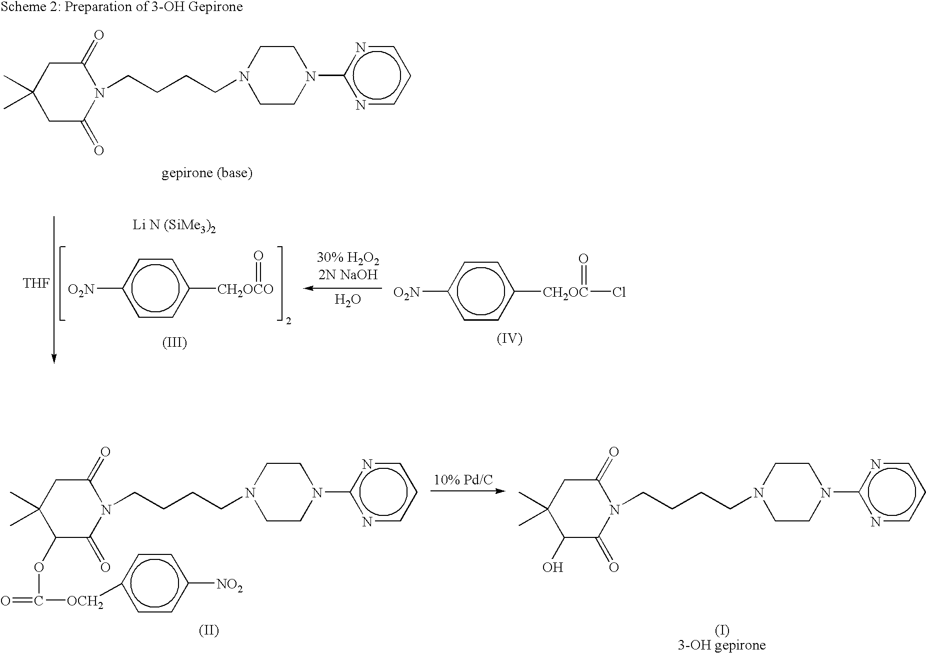

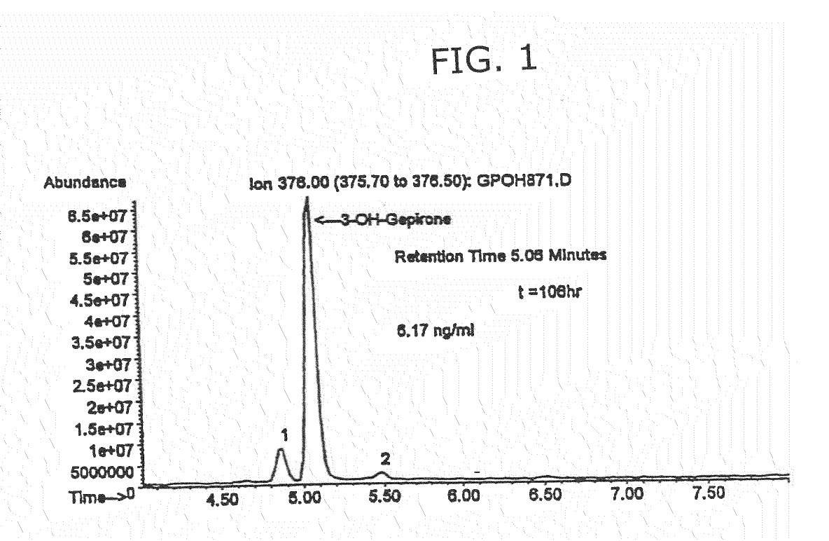

This method of preparation is provided as a helpful example and illustrates a convenient synthesis of 3-OH gepirone. A method in van Molke, et al., Psychopharmacology, 140: 293-299 (1998), specifically incorporated herein by reference, can be used to produce 3-OH gepirone and the other bioactive metabolites of gepirone by enzymatic (human or rat liver microsomes) conversion of gepirone in vitro. Isolation or purification of the 3-OH gepirone compounds can be by the method described in FIG. 1 or other methods known in the art (see Odontiadis, J. Pharmaceut. Biomedical Analysis 1996 14:347-351, specifically incorporated herein by reference).

Systemic administration may be accomplished by administration of a precursor or prodrug form of 3-OH gepirone (e.g., gepirone) to mammals, resulting in systemic introduction of 3-OH gepirone.

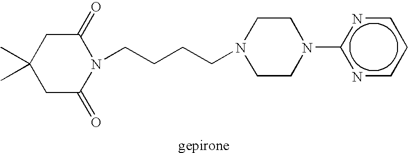

Methods of assessing the receptor (5-HT_{1A} and dopamine) agonistic activity of the compounds of the present invention are well known to those of skill in the art. These methods can be found in Iser-Strenegr, et al (Brain Res 1986 November; 395(t):57-65), Millan, et al (J Pharmacol Exp Ther 1993 March; 264(3):1364-76), and Perrone R, et al (J Med Chem 1995 Mar. 17; 38(6):942-9), all of which are incorporated herein by reference.

A drug that treats attention deficit disorder is assessed by measuring the child's behavior before and after treatment with a drug of the present invention. Measurements of the child's behavior include clinical measures and rating scales. Two clinical measures are the simulated classroom (Gadow et al, Stony Brook, N.Y.: Checkmate Plus, 1996) and the continuous performance test (Roberts et al, J Pediatr Psychol 1984; 9:177-191, Halperin et al, J Am Acad Child Adolesc Psychiatry 1992; 31:190-196, and Halperin et al, J Am Acad Child Adolesc Psychiatry 1988; 27:326-329).

The simulated classroom requires the child to sit alone at a desk in a small classroom completing work, and not playing with toys on an adjacent table. Clinic sessions are video-recorded through a one-way window to facilitate ease of scoring. The 3 ADHD behaviors measured are Off Task, Fidgeting, and Worksheets (number of items completed correctly). The continuous performance test (CPT) requires a child to press the space bar whenever the letter “A” followed the letter “X” on a computer screen. The CPT generates 3 scores (inattention, impulsivity, and dyscontrol) and takes approximately 12 minutes to complete. Examples of rating scales include the Abbreviated Teacher Questionnaire (ATQ; Conners, Psychopharm Bull 1973; 9:24-84 and Epstein et al, J Special Educ 1986; 20:219-229), the Iowa-Corners Teachers Rating Scale (Loney et al, Advances in developmental and behavioral pediatrics 1982; vol. 3, Greenwich, CT:JAI Press; 113-147), and the Primary Secondary Symptom Checklist (Loney, Poster presented at the annual meeting of the American Psychological Association, Toronto, Ontario, 1984). Normally both teachers and parents scales are rated.

The results of the pre-treatment and post-treatment evaluations can be analyzed by appropriate statistical procedures, such as those contained in Mandel, The Statistical Analysis of Experimental Data, Dover Publications; Toronto, Ontario, 1964.

One skilled in the art is familiar with numerous methods for designing and optimizing formulations and delivery methods to deliver 5-HT_{1A} receptor agonists, in particular gepirone, ipsapirone, tandospirone, flesinoxan, and adatanserin in effective and non-toxic ways. Remington's Pharmaceuticals Sciences, 18th Edition (specifically incorporated herein by reference), can be relied on and used for these purposes, especially Part 8 therein, “Pharmaceutical Preparations and Their Manufacture.” The following compounds, compositions, delivery methods, delivery dosages, and formulations are specifically envisioned as suitable for, but not meant to limit, the present invention.

The pharmaceutical compounds suitable for administration in the present invention may be hydrochloride salts, but the free bases and other pharmaceutically acceptable salts are also suitable. The term “pharmaceutically acceptable salt” is well known in the art, as described in S. M. Berge, et al. (J Pharmaceutical Sciences, 66: 1-19, 1977). Suitable pharmaceutically acceptable salts for administration in the present invention include acid addition salts. The acid addition salt may be formed by mixing a solution of the compound with a solution of a pharmaceutically acceptable non-toxic acid such as hydrochloric acid, hydrobromic acid, fumaric acid, maleic acid, succinic acid, acetic acid, citric acid, tartaric acid, carbonic acid, phosphoric acid, perchloric acid, sulphuric acid, oxalic acid, or malonic acid. Where the compound carries an acidic group, for example a carboxylic acid group, the present invention also contemplates salts thereof, preferably nontoxic pharmaceutically acceptable salts thereof, such as the sodium, potassium and calcium salts thereof.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, furnarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pictate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, salts of amine groups. Salts of amine groups may also comprise the quaternary ammonium salts in which the amino nitrogen atom carries an alkyl, alkenyl, alkynyl or aralkyl group, nontoxic ammonium, quaternary ammonium, and amine cations formed using counterions such as halide, hydroxide, carboxylate, sulfate, phosphate, nitrate, loweralkyl sulfonate and aryl sulfonate.

A therapeutically effective amount of the pharmaceutical compounds suitable for administration in the present invention may be administered alone or in combination with one or more pharmaceutically acceptable carriers. As used herein, the term “pharmaceutically acceptable carrier” means a non-toxic, inert solid, semi-solid or liquid filer, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions suitable for administration in the invention can be administered to patients in need thereof orally, rectally, nasally, parenterally (e.g., intramuscular, intraperitoneal, intravenous or subcutaneous injection, or implant), intracisternally, intravaginally, intraperitoneally, sublingually, topically (e.g., as a powder, ointment, or drop), bucally, as an oral spray, or a nasal spray. The pharmaceutical compositions can be formulated in dosage forms appropriate for each route of administration.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art. The inert diluents may include, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. The liquid dosage form for oral administration may also contain adjuvants, which include wetting agents, emulsifying and suspending agents, sweetening, flavoring, and perfuming agents. Other dosage forms for oral administration include, for example, aqueous suspensions containing the active compound in an aqueous medium in the presence of a non-toxic suspending agent such as sodium carboxy-methylcellulose, and oily suspensions containing a compound of the present invention in a suitable vegetable oil, for example arachis oil.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This maybe accomplished by the use of a liquid suspension of crystalline or amorphous material with poor water solubility. The rate of absorption of the drug then depends upon its rate of dissolution, which, in turn, may depend upon crystal size and crystalline form. Alternatively, dissolving or suspending the drug in an oil vehicle accomplishes delayed absorption of a parenterally administered drug form. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, prills, powders, and granules. In such solid dosage forms, the active compound is mixed with at least one inert, pharmaceutically acceptable excipient or carrier. In addition, the solid dosage form may contain one or more fillers, extenders, binders, humectants, disintegrating agents, retarding agents, absorption accelerators, wetting agents, absorbents, or lubricants. Examples of suitable fillers or extenders include, starches, lactose, sucrose, glucose, mannitol, and silicic acid, sodium citrate and dicalcium phosphate. Examples of suitable binders include, microcrystalline cellulose, carboxymethylcellulose, alginates, gelatin, polyvinylpyrrolidinone, sucrose, and acacia. Glycerol is an example of a suitable humectant. Examples of suitable disintegrating agents include, agar-agar, calcium carbonate, potato or tapioca starch, maize starch, alginic acid, certain silicates, and sodium carbonate. Paraffin is an example of a suitable solution-retarding agent. As absorption accelerators, any quaternary ammonium compound may be used. Examples of suitable wetting agents include, cetyl alcohol and glycerol monostearate. Examples of suitable absorbents include, kaolin and bentonite clay. Examples of suitable lubricants include, talc, calcium stearate, magnesium stearate, solid polyethylene glycols, sodium lauryl sulfate. In the case of capsules, tablets and pills, the dosage form may also comprise buffering agents.

The tablets may, if desired, be coated using known methods and excipients that may include enteric coating using for example hydroxypropylmethylcellulose phthalate. The tablets may be formulated in a manner known to those skilled in the art so as to give a sustained release of the compounds of the present invention. Such tablets may, if desired, be provided with enteric coatings by known methods, for example by the use of cellulose acetate phthalate. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Similarly, capsules, for example hard or soft gelatin capsules, containing the active compound with or without added excipients, may be prepared by known methods and, if desired, provided with enteric coatings in a known manner. The contents of the capsule may be formulated using known methods so as to give sustained release of the active compound. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hardfilled gelatin capsules using such excipients as lactose or milk sugar as well as high molecular weight polyethylene glycols.

If desired, the compounds of the present invention can be incorporated into slow release or targeted delivery systems such as polymer matrices, liposomes and microspheres. They may be sterilized, for example, by filtration through a bacteria-retaining filter, or by incorporating sterilizing agents in the form of sterile solid compositions that can dissolve in sterile water, or some other sterile injectable medium immediately before use.

The active compound may be formulated into granules with or without additional excipients. The granules may be ingested directly by the patient or they may be added to a suitable liquid carrier (for example, water) before ingestion. The granules may contain disintegrates, e.g. an effervescent couple formed from an acid and a carbonate or bicarbonate salt to facilitate dispersion in the liquid medium.

Dosage forms for topical or transdermal administration of a compound of this invention include ointments, pastes, creams, lotions, gels, powders, solutions, sprays, inhalants or patches. Transdermal patches have the added advantage of providing controlled delivery of a compound to the body. The rate can be controlled by either providing a rate controlling membrane or by dispersing the compound in a polymer matrix or gel. The active component is admixed under sterile conditions with a pharmaceutically acceptable carrier and any needed preservatives or buffers as may be required. Dissolving or dispensing the compound in the proper medium can make such dosage forms. Absorption enhancers can also be used to increase the flux of the compound across the skin. Ophthalmic formulation, eardrops, eye ointments, powders and solutions are also contemplated as being within the scope of this invention.

Dosage forms for topical administration may comprise a matrix in which the pharmacologically active compounds of the present invention are dispersed so that the compounds are held in contact with the skin in order to administer the compounds transdermally. A suitable transdermal composition may be prepared by mixing the pharmaceutically active compound with a topical vehicle, such as animal and vegetable fats, oils, petrolatum, waxes, paraffins, starch, tragacanth, cellulose derivatives, polyethylene glycols, silicones, bentonites, silicic acid, talc and zinc oxide, or mixtures thereof, together with a potential transdermal accelerant such as dimethyl sulphoxide or propylene glycol. Alternatively the active compounds may be dispersed in a pharmaceutically acceptable paste, cream, gel or ointment base. The amount of active compound contained in a topical formulation should be such that a therapeutically effective amount of the compound is delivered during the period of time for which the topical formulation is intended to be on the skin.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. The therapeutically active compound may be formulated into a composition, which is dispersed as an aerosol into the patient's oral or nasal cavity. Such aerosols may be administered from a pump pack or from a pressurized pack containing a volatile propellant.

The therapeutically active compounds used in the method of the present invention may also be administered by continuous infusion either from an external source, for example by intravenous infusion or from a source of the compound placed within the body. Internal sources include implanted reservoirs containing the compound to be infused which is continuously released for example by osmosis and implants which may be (a) liquid such as an oily suspension of the compound to be infused for example in the form of a very sparingly water-soluble derivative such as a dodecanoate salt or a lipophilic ester or (b) solid in the form of an implanted support, for example of a synthetic resin or waxy material, for the compound to be infused. The support may be a single body containing the entire compound or a series of several bodies each containing part of the compound to be delivered. The amount of active compound present in an internal source should be such that a therapeutically effective amount of the compound is delivered over a long period of time.

It will be known to those skilled in the art that there are numerous compounds, which may be used for treating attention deficit disorder, anxiety, depression, or sexual dysfunction in a patient. Combinations of these therapeutic agents, some of which have also been mentioned herein, will bring additional, complementary, and often synergistic properties to enhance the desirable properties of these various therapeutic agents. In these combinations, the 5-HT_{1A} agonist and the therapeutic agents may be independently present in dose ranges from one one-hundredth to one times the dose levels which are effective when these compounds are used singly. In such combination therapy, the 5-HT_{1A} agonist may be administered with the other therapeutic agent (e.g., concurrently, concomitantly, sequentially, or in a unitary formulation) such that their therapeutic efficacy overlaps.

The 5-HT_{1A} agonist may be employed in conjunction with an agent selected from the group consisting of stimulants, hypnotics, anxiolytics, antipsychotics, antianxiety agents, minor tranquilizers, benzodiazepines, barbituates, serotonin agonists, selective serotonin reuptake inhibitors, dopamine antagonists, 5-HT_{1A} agonists, 5-HT₂ antagonists, non-steroidal anti-inflammatory drugs, monoamine oxidase inhibitors, muscarinic agonists, norephinephrine uptake inhibitors, essential fatty acids, and neurokinin-1 receptor antagonist.

For example, for treating attention deficit disorder in a patient a 5-HT_{1A} agonist may be given in combination with such compounds as: adinazolam, allobarbital, alonimid, alprazolam, amitriptyline, amobarbital, amoxapine, bentazepam, benzoctamine, brotizolam, bupropion, busprione, butabarbital, butalbital, caffeine, capuride, carbocloral, chloral betaine, chloral hydrate, chlordiazepoxide, clomipramine, cloperidone, clorazepate, clorethate, clozapine, cyprazepam, deanol, desipranune, dexclamol, dextroamphetamine, diazepam, dichloralphenazone, divalproex, diphenhydramine, doxepin, duloxetine, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, methylphenidate (including d-methylphenidate, especially d-methylphenidate hydrochloride), midaflur, midazolam, nefazodone, nisobamate, nitrazepam, nortriptyline, omega-3 fatty acids, oxazepam, paraldehyde, paroxetine, pemoline, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, xanomeline, zaleplon, zolazepam, zolpidem, and salts thereof, and combinations thereof, and the like, as well as admixtures and combinations thereof.

As used herein, the term “therapeutically effective amount” refers to that amount of a compound or preparation of the present invention that successfully prevents or reduces the severity of symptoms associated with attention deficit disorder, with or without hyperactivity. This term also embraces the amount of a compound or preparation of the present invention that successfully prevents or reduces the severity of symptoms associated with attention deficit disorder, with or without hyperactivity, anxiety, depression, and/or sexual dysfunction when the patient also suffers from anxiety, depression, obesity, drug abuse/addiction, alcohol abuse, sleep disorders, TIC disorder, or behavioral/cognitive symptoms of Alzheimer's disease.

As used herein, the term “TIC disorder” refers to a one or more disorders, which include Tourette's Disorder, Chronic Motor or Vocal Tic Disorder, Transient Tic Disorder, and Tic Disorder Not Otherwise Specified. A more complete description of the symptoms and diagnostic criteria of TIC disorder is provided by DSM-IV (Diagnostic and Statistical Manual of Mental Disorders, 1994; 100-105), which is incorporated herein by reference.

It is contemplated that the therapeutically effective amount of a composition will depend on a number of factors, including by not limited to the age of the patient, immune status, race, and sex of the patient, and the severity of the condition/disease, and the past medical history of the patient, and always lies within the sound discretion of the administering physician. Generally, the total daily dose of the compounds of this invention administered to a patient in single or in divided doses can be in amounts, for example, 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day). Single dose compositions may contain such amounts or submultiples thereof to make up the daily dose. In general, treatment regimens according to the present invention comprise administration to a patient in need of such treatment 0.25-0.75 mg/kg of body weight/day of gepirone (approximately 15 mg/day), 0.003-0.06 mg/kg of body weight/day of flesinoxan (approximately 0.4 mg/day), and 0.5-3.0 mg/kg of body weight/day of adatanserin (approximately 120 mg/day) in single or multiple doses. In addition, the total daily dose of the azapirone compounds, having no dopamine receptor activity (including ipsapirone and tandospirone), of this invention administered to a patient in need thereof, in single or in divided doses can be in amounts of 0.25-3.0 mg/kg of body weight/day.

Treatment regimens according to the present invention also include concurrently administering to a patient in need thereof mixtures, in single or divided doses, of two or more of the compounds of the present invention. When the compounds of the present invention are administered concurrently as mixtures, the therapeutically effective amount to be administered lies within the sound discretion of the administering physician; preferably, the compounds of the present invention may be administered to a patient in single or in divided doses in amounts of, for example, 0.25-0.75 mg/kg of body weight/day of gepirone (approximately 15 mg/day); 0.003-0.06 mg/kg of body weight/day of flesinoxan (approximately 0.4 mg/day), 0.5-3.0 mg/kg of body weight/day of adatanserin (approximately 120 mg/day), and 0.25-3.0 mg/kg of body weight/day of ipsapirone and tandospirone.

Alternatively, treatment regimens according to the present invention include sequentially administering to a patient in need thereof, in single or divided doses, two or more of the compounds of the present invention. An example of a sequential administration strategy includes administering a therapeutically effective amount of a first compound followed by, on the same day or a subsequent day, a single or divided dose of a therapeutically effective amount of one or more additional compounds. As used herein, the term “subsequent day” refers to any day ranging from the next day (>24 hours) to one week (≦168 hours) after administration of the previous compound. The term “same day” refers to any time frame ranging from immediately after administration of the previous compound to ≦24 hours after administration of the previous compound.

When the compounds of the present invention are administered sequentially as a part of a combination therapy, the therapeutically effective amount to be administered lies within the sound discretion of the administering physician; preferably, the compounds of the present invention may be administered to a patient in single or in divided doses in amounts of, for example, 0.25-0.75 mg/kg of body weight/day of gepirone (approximately 15 mg/day); 0.003-0.06 mg/kg of body weight/day of flesinoxan (approximately 0.4 mg/day), 0.5-3.0 mg/kg of body weight/day of adatanserin (approximately 120 mg/day), and 0.25-3.0 mg/kg of body weight/day of ipsapirone and tandospirone.

As used herein, the terms “treat”, “treating”, and “treatment” also embrace the terms alleviation and amelioration. In addition, it is also within the scope of the present invention to use the methods described and/or claimed herein for the prevention of attention deficit disorder, with or without hyperactivity, as well as the symptoms associated therewith. Moreover, the terms “treat”, “treating”, and “treatment” also may embrace prevention of attention deficit disorder.

Having generally described this invention, a further understanding can be obtained by reference to certain specific examples, which are provided herein for purposes of illustration only, and are not intended to be limiting unless otherwise specified.

### Description of Exemplary and Specific Embodiments

The uses of the compounds that constitute this invention and the methods of preparation will appear more fully in light of the following examples, which are given for the purpose of illustration only and are not to be construed as limiting the invention in sphere or scope. All of the references referred to in this specification, for whatever purpose, can be used and relied on to make and used specific embodiments of the invention. Thus, all of the references are specifically incorporated into this disclosure by reference.

### Example 1

### Preparation of 3-OH Gepirone (I)

A. Di-4-nitrobenzyl peroxydicarbonate (III) Di-4-nitrobenzyl peroxydicarbonate was prepared using a modification of the literature procedure (Strain, et al., J Am. Chem. Soc., 1950, 72:1254; specifically incorporated herein by reference). Thus, to an ice-cold solution of 4-nitrobenzyl chloroformate (10.11 g, 4.7 mmol) in acetone (20 mL) was added dropwise over 30 min an ice-cold mixture of 30% H₂O₂ (2.7 mL, 24 mmol) and 2.35 N NaOH (20 mL, 47 mmol). The mixture was vigorously stirred for 15 min and then it was filtered and the filter-cake was washed with water and then with hexane. The resulting damp solid was taken up in dichloromethane, the solution was dried (Na₂SO₄) and then it was diluted with an equal volume of hexane. Concentration of this solution at 20° C. on a rotary evaporator gave a crystalline precipitate which was filtered, washed with hexane and dried in vacuo to give compound III (6.82 g, 74%) as pale yellow microcrystals, mp 104° C. (dec).

Di-4-nitrobenzyl peroxydicarbonate was found to be a relatively stable material, which decomposed as its melting point with slow gas evolution. In comparison, dibenzyl peroxydicarbonate (Cf Gore and Vederas, J. Org. Chem., 1986, 51:3700; specifically incorporated herein by reference) decomposed with a sudden vigorous expulsion of material from the melting point capillary.

### B. 4,4-Dimethyl-3-(4-nitrobenzyloxycarbonyloxy)-1-[4-[4-(2-pyrimidinyl)-1-piperazinyl]butyl]-2.6-piperidinedione (II)

To a solution of 4-dimethyl-1-[4-[4-(2-pyrimidinyl)-1-piperazinyl]butyl]-2,6-piperidinedione (gepirone: 12.7 g, 356 mmole) in dry THF (200 mL) was added LiN (Me₃Si)₂ (37.3 mL of a 1 M THF solution) at −78° C. and the mixture was stirred for 2.5 h. A solution of di-4-nitrobenzyl peroxydicarbonate (15 g) in dry THF (100 mL) was then added dropwise over 1 h. Stirring was continued at −78° C. for an additional 2 h.

The cooling bath was then removed and the reaction solution was poured into a mixture of H₂O and EtOAc. The organic phase was separated and washed with H₂O and then with brine. The organic phase was dried and then evaporated to a brown gum. Flash chromatography of the gum, eluting the silica gel column with EtOAc, gave crude product which was titrated in hexane to provide 7.5 g (58%) product (II) with recovery of 2.5 g of gepirone after elution of the column with acetone.

### C. 4,4-Dimethyl-3-hydroxy-1-[4-[4-(2-pyrimidinyl)-1-piperazinyl]butyl]-2.6-piperidinedione (I: 3-OH gepirone)

A mixture of II (7.0 g; 12.6 mmole) and 10% Pd/C (3.5 g) in MeOH (70 mL) was hydrogenated in a Parr shaker at 30 psi for 0.5 h. The hydrogenation mixture was filtered through a Celite pad, which was then washed with THF. The filtrate was evaporated to a gum which was solidified by titration in ether. Filtration gave 2 g of crude product as a beige solid. The filtrate was evaporated and the residue was flash chromatographed through a silica column eluting with EtOAc to provide an additional 1 g of crude product. The crude product was combined and suspended in MeOH. A small portion of ether was added and the mixture was filtered to give 2.5 g of I (3-OH gepirone) as a white solid. This material was recrystallized (acetone-hexane) to give a solid mp 122-124° C. (gas evolution).

### Example 2

### Comparison of 3-OH Gepirone and Gepirone Metabolites to Ggeplirone

As a basis for estimating the bioavailability of potential therapeutic compounds, a number of octanol-water partition coefficient calculations have been used (see Poole, J. of Chromatography B, 745:117-126 (2000); Ishizaki, J. Pharm. Pharmacol., 49:762-767 (1997) (each specifically incorporated herein by reference)). Using these partition coefficients, the bioavailability of gepirone metabolites can be calculated.

| | log P_{ow} Partition Coefficient Octanol-Water | | |
|---|---|---|---|
| | Crippen | Viswanadhan's | Broto's |
| Compound | fragmentation | fragmentation | fragmentation |
| gepirone | 1.38 ± 0.47 | 1.32 ± 0.49 | 1.13 ± 0.97 |
| 3-OH gepirone | 0.73 ± 0.47 | 0.89 ± 0.49 | −0.23 ± 1.11 |

In all methods, 3-OH gepirone possesses higher water solubility (lower log P_{OW}) and lower lipid-solubility as compared to gepirone.

The short half-life characteristics of gepirone can be attributed to its high lipid solubility, which makes it much more susceptible to first-pass degradation by the liver. Since 3-OH gepirone is less soluble in lipid, its first pass degradation characteristics will result in a much longer half-life in plasma. Furthermore, the range in lipid solubility for 3-OH gepirone (about 5:1 to 8:1), when the Broto calculation is discarded because of the high standard deviation, is within that generally accepted as appropriate for psychoactive compounds that may interact within receptors in the brain. Accordingly, 3-OH gepirone possesses superior characteristics from the standpoint of an immediate acting pharmaceutical compound that avoids the first-pass degradation by the liver.

### Example 3

### Dosage of 3-OH Gepirone

The 3-OH gepirone compositions and dosage forms of the invention are designed to deliver an effective anxiolytic, anti-depressant, or psychoactive amount of 3-OH gepirone or a pharmaceutically acceptable salt thereof to a mammal, preferably a human. Effective doses of about 0.01 to 40 mg/kg body weight are contemplated, preferred ranges are about 0.1 to about 2 mg per kg body weight. For certain central nervous system disorders, 15 to 90 mg/day, preferably 30-60 mg/day, are recommended. See U.S. Pat. No. 4,771,053 to Cott et al. (specifically incorporated herein by reference). Administration of bioactive gepirone metabolites according to the present invention may be made by the parenteral, oral, buccal, rectal, or transdemmal routes. The oral route is preferred, however. The clinical dosage range for alleviation of major depressive disorders is expected to be less than about 100 mg per day, generally in the 15 to 90 mg range, and preferably in the range of 30-60 mg per day. Since the dosage should be tailored to the individual patient, the usual practice is to commence with a dose of about 5 mg administered once, twice, or three times per day and then to increase the dose every 2 or 3 days by 5 mg at each dosage time until the desired response is observed or until the patient exhibits side effects. A single daily dosage may be applicable, but division of the daily dose into 2 or 3 portions is also possible. One skilled in the art is familiar with methods and techniques to optimize an effective dose and minimize toxic and adverse effects in a dose. One can rely on methods and techniques known in the art (See Remington's Pharmaceutical Sciences, Genero, et al. eds., 18th Edition, Easton: Mack Publishing Co.; U.S. Pat. Nos. 4,782,060, 4,771,053, 5,478,572, and 5,468,749, each specifically incorporated herein by reference).

### Example 4

### Purification of Bioactive Gepirone Metabolites

As noted above, 3-OH gepirone can be prepared by chemical synthesis or enzymatic methods. Purification of 3-OH gepirone from either method can be achieved with HPLC methods using conventional techniques known in the art. The other bioactive gepirone metabolites can be prepared in similar ways.

In FIG. 1, the purified gepirone metabolites are separated by HPLC using conditions as described below. Peaks showing 3-OH gepirone and 5-OH gepirone are identified in Figure, demonstrating the effectiveness of HPLC separation with C18 columns. The data in FIG. 1 was prepared using an electrospray-HPLC/MS analysis of a 10 ul sample from plasma. A linear gradient of 95% buffer A to 50% buffer A in 8.0 minutes was used (buffer A is aqueous 750 uM ammonium formate and mobile phase B is 80:20 acetonitrile:water (acidified with 0.15% formic acid)). A Luna 5u C18 (2) 150.times.1.0 mm HPLC column was used (Phenomenex).

### Example 5

### Determination of 3-OH Gepirone Concentrations in Plasma

FIG. 2 shows the concentration of 3-OH gepirone in plasma of human subjects. Each sample corresponds to a 0.5 ml plasma sample, extracted with 6 ml of (2:1) (v/v) hexane:chloroform for 1 hour. After separation by centrifuge, the organic layer is transferred to a 10 ml conical tube and 90 ul of 1% formic acid is added. The tube is vortexed for 10 minutes and centrifuged for 5 minutes. About 80 ul of the formic acid layer is transferred into injection vials for HPLC/MS analysis. The electrospray-HPLC/MS system noted in Example 4 above, as described for FIG. 1, can be used to determine the levels of 3-OH gepirone.

### Example 6

### The Benefit of 5-HT_{1A} Partial Agonists in ADHD Alone

Experiments to gain FDA approval for this indication would involve two well-controlled, well-designed trials of the test drug in patients suffering with ADHD. A typical study would involve 50-100 children allocated 50% to the test drug and 50% to placebo. Medication would be given daily for approximately 8 weeks. Assessment of severity of ADHD symptoms would be completed prior to drug treatment and at regular intervals throughout the 8 weeks. The measurements and ratings would be similar to those mentioned above. Appropriate statistical procedures would be applied to the results. The study would be similar to that conducted by Greenhill et al (Pediatrics 2002; 109:E39-52).

### Example 7

### The Benefit of 5-HT_{1A} Partial Agonists in Children with ADHD and TIC Disorder

The procedure here would be similar to Example 6. An experiment similar to that provided by Gadow et al (J Clin Psychopharm 2002; 22:267-274) for methylphenidate (RITALIN™) could also be utilized.

### Example 8

### The Benefit of 5-HT_{1A} Partial Agonists in Children with ADHD and Anxiety Symptoms

The procedure here would be similar to Example 6. An experiment similar to those provided by Taylor et al (Psychol Med 1987; 17:121-143) and/or Pliszka (J Am Acad Child Adolesc Psychiatry 1989; 28:882-7) could also be utilized.

### Example 9

### The Benefit of 5-HT_{1A} Partial Agonist in Children with ADHD and Depressive Symptoms

The procedure here would be similar to Example 6. Additional diagnoses of depression would be made by DSM-IV criteria, and rating scales would include the Hamilton Depression Rating Scale (M. Hamilton, J Neurol Neurosurg Psychiatry 1960; 23:56-62).

The bioactive gepirone metabolites exemplified by formula (I), 3-OH gepirone, are useful psychotropic agents, which exhibit selective anxiolytic and antidepressant action. In particular, these improved compounds appear to offer an advantage over buspirone and its close analogs in that antipsychotic or neuroleptic action, with its potential adverse side effects, appears markedly reduced or absent. This realizes one objective of the instant invention, i.e., to increase selectively for this class of antidepressant and anxiolytic agents. Various in vivo and in vitro animal tests confirm that while the formula (I) compounds exhibit little antipsychotic activity, they otherwise retain or improve upon the novel anxioselective and anditdepressant profile exhibited by buspirone and its close analogs.

The examples and description above are exemplary and should not be taken as a limitation to the scope of the invention or the claims that follow. One skilled in the art is familiar with a variety of techniques to deduce and test variations or derivatives of the methods, compositions, and formulations described that fall within the scope of this invention. Preparing and using these variations or derivatives is enabled by this specification in the hands of those skilled in the art.

1. A method of treating attention deficit disorder, or symptoms thereof, in a patient in need thereof comprising administering a therapeutically effective amount of 3-OH gepirone, or a pharmaceutically acceptable salt or hydrate thereof, to the patient.

2. The method of claim 1, wherein the attention deficit disorder in the patient is further associated with hyperactivity.

3. The method of claim 1, wherein the 3-OH gepirone is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

4. The method of claim 1, wherein the 3-OH gepirone is administered with methylphenidate.

5. The method of claim 1, wherein the 3-OH gepirone is administered with a pharmaceutically acceptable carrier.

6. The method of claim 1, wherein said administering is selected from the group consisting of oral, rectal, nasal, parenteral, intracisternal, intravaginal, intraperitoneal, sublingual, topical, and bucal.

7. The method of claim 6, wherein said administering is oral or parenteral.

8. The method of claim 1, wherein the therapeutically effective amount of the 3-OH gepirone is about 0.1 to about 2 mg per kg of body weight per day.

9. The method of claim 1, wherein the bioactive gepirone metabolite is present in the plasma of the mammal at about 1 to about 5 ng/ml within two hours of administration.

10. The method of claim 1, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

11. The method of claim 1, wherein the patient in need thereof also suffers from one or more disorders selected from the group consisting of anxiety, depression, and TIC disorder.

12. A method of treating attention deficit disorder, or symptoms thereof, in a patient in need thereof comprising administering a therapeutically effective amount of two or more compounds selected from the group consisting of 3-OH gepirone, ipsapirone, tandospirone, flesinoxan, and adatanserin.

13. The method of claim 12, wherein the attention deficit disorder in the patient is further associated with hyperactivity.

14. The method of claim 12, wherein the compounds are administered with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

15. The method of claim 12, wherein the compounds are administered with a pharmaceutically acceptable carrier.

16. The method of claim 12, wherein said administering is selected from the group consisting of oral, rectal, nasal, parenteral, intracisternal, intravaginal, intraperitoneal, sublingual, topical, and bucal.

17. The method of claim 16, wherein said administering is oral or parenteral.

18. The method of claim 12, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

19. The method of claim 12, wherein the patient in need thereof also suffers from one or more disorders selected from the group consisting of anxiety, depression, and TIC disorder.

20. The method of claim 12, wherein said two or more compounds are administered concurrently.

21. The method of claim 12, wherein said two or more compounds are administered sequentially.

22. The method of claim 21, wherein said two or more compounds are administered on the same day.

23. The method of claim 21, wherein said two or more compounds are administered on subsequent days.

24. A method of treating sexual dysfunction, or symptoms thereof, in a patient in need thereof comprising administering a therapeutically effective amount of 3-OH gepirone, or a pharmaceutically acceptable salt or hydrate thereof, to the patient.

25. The method of claim 24, wherein the 3-OH gepirone is administered in conjunction with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

26. The method of claim 24, wherein the 3-OH gepirone is administered with methylphenidate.

27. The method of claim 24, wherein the 3-OH gepirone is administered with a pharmaceutically acceptable carrier.

28. The method of claim 24, wherein said administering is selected from the group consisting of oral, rectal, nasal, parenteral, intracisternal, intravaginal, intraperitoneal, sublingual, topical, and bucal.

29. The method of claim 28, wherein said administering is oral or parenteral.

30. The method of claim 24, wherein the therapeutically effective amount of the 3-OH gepirone is about 0.1 to about 2 mg per kg of body weight per day.

31. The method of claim 24, wherein the bioactive gepirone metabolite is present in the plasma of the mammal at about 1 to about 5 ng/ml within two hours of administration.

32. The method of claim 24, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

33. The method of claim 24, wherein the patient in need thereof also suffers from one or more disorders selected from the group consisting of anxiety, depression, and TIC disorder.

34. A method of treating sexual dysfunction, or symptoms thereof, in a patient in need thereof comprising administering a therapeutically effective amount of two or more compounds selected from the group consisting of 3-OH gepirone, ipsapirone, tandospirone, flesinoxan, and adatanserin.

35. The method of claim 34, wherein the compounds are administered with at least one agent selected from the group consisting of a stimulant, a hypnotic, an anxiolytic, an antipsychotic, an antianxiety agent, a minor tranquilizer, a benzodiazepine, a barbituate, a serotonin agonist, a selective serotonin reuptake inhibitor, a dopamine antagonist, a 5-HT_{1A} agonist, a 5-HT₂ antagonist, a non-steroidal anti-inflammatory drug, a monoamine oxidase inhibitor, a muscarinic agonist, a norephinephrine uptake inhibitor, an essential fatty acid, and a neurokinin-1 receptor antagonist.

36. The method of claim 34, wherein the compounds are administered with a pharmaceutically acceptable carrier.

37. The method of claim 34, wherein said administering is selected from the group consisting of oral, rectal, nasal, parenteral, intracisternal, intravaginal, intraperitoneal, sublingual, topical, and bucal.

38. The method of claim 37, wherein said administering is oral or parenteral.

39. The method of claim 34, wherein the patient in need thereof also suffers from one or more disorders selected from the group consisting of anxiety, depression, obesity, drug abuse/addiction, alcohol abuse, sleep disorders, TIC disorder, and behavioral/cognitive symptoms of Alzheimer's disease.

40. The method of claim 34, wherein the patient in need thereof also suffers from one or more disorders selected from the group consisting of anxiety, depression, and TIC disorder.

41. The method of claim 34, wherein said two or more compounds are administered concurrently.

42. The method of claim 34, wherein said two or more compounds are administered sequentially.

43. The method of claim 42, wherein said two or more compounds are administered on the same day.

44. The method of claim 42, wherein said two or more compounds are administered on subsequent days.
